(19) United States
(12) Patent Application Publication
(10) Pub. No.: US 2015/0142372 A1
(43) Pub. Date: May 21, 2015

(54) PROSTHETIC PLACEMENT TOOL AND ASSOCIATED METHODS

(71) Applicant: Polaris Surgical, LLC, Atlanta, GA (US)

(72) Inventor: Angad Singh, Marietta, GA (US)

(73) Assignee: Polaris Surgical, LLC, Atlanta, GA (US)

(21) Appl. No.: 14/084,119

(22) Filed: Nov. 19, 2013

(51) Int. Cl. A61F 2/46; A61B 5/107; G01B 21/16

(52) U.S. Cl. CPC A61F 2/4657; G01B 21/16; A61B 5/1071; A61F 2002/4668
USPC 702/150

(57) ABSTRACT

A method for estimating an orientation for placement of a prosthetic implant comprises receiving information indicative of a first virtual axis established between estimated positions of two anatomic landmarks in the anatomical area of interest. The method also comprises calculating an orientation of a first virtual plane, the first virtual plane being perpendicular to the first virtual axis. The method further comprises receiving information indicative of a second virtual axis established between at least one of the estimated positions of the first two landmarks and a third anatomic landmark in the anatomical area of interest. The method also comprises calculating an orientation of a second virtual plane based, at least in part, on the first virtual axis and the second virtual axis. The method further comprises estimating an angle between the orientation sensor and at least one of the first virtual plane or the second virtual plane.

### TECHNICAL FIELD

The present disclosure relates generally to orthopedic surgery and, more particularly, to an apparatus and method for intra-operatively measuring prosthetic placement parameters during orthopedic arthroplastic procedures.

### BACKGROUND

Orthopedic procedures involving resurfacing, replacement, or reconstruction of joints using multi component prosthesis with articulating surfaces. In such procedures proper placement of the prosthetic component is critical for longevity of the implant, positive clinical outcomes, and patient satisfaction. For example, ball-and-socket joints, such as the hip, typically involve replacement of a “socket” portion of a bone of the joint with a prosthetic cup. In such procedures, proper placement of the prosthetic cup is imperative to restoring normal joint function. Indeed, improper placement of the prosthetic cup can lead to a number of problems, such as subluxation, dislocation, and/or femoral impingement, each of which inhibit proper joint function, increases patient discomfort, and potentially lead to painful and costly corrective/revision procedures.

Currently, many orthopedic surgeons intra-operatively evaluate prosthetic component placement using an imprecise combination of subjective experience of the surgeon and rudimentary mechanical instrumentation. Prosthetic placement parameters such as version (e.g. anteversion/retroversion) and inclination (e.g. abduction/adduction) may be manually estimated by the surgeon, using rough and imprecise (i.e., “eyeball”) estimating methods or mechanical guides/jigs. This process for intra-operative evaluation is extremely subjective and imprecise, and the performance of the reconstructed joint is highly variable and dependent on the experience level of the surgeon. Perhaps not surprisingly, it is difficult for patients and doctors to reliably predict the relative success of the surgery (and the need for subsequent corrective/adjustment surgeries) until well after the initial procedure. Such uncertainty has a negative impact on long term clinical outcomes, patient quality of life, and the ability to predict and control costs associated with surgery, recovery, and rehabilitation.

Some computer/robotically-assisted surgical systems provide a platform for more reliably estimating prosthetic placement parameters. These systems typically require complex and sophisticated tracking equipment, bulky markers/sensors, time-consuming instrument calibration/registration procedures, and highly-specialized software packages that often require technical support personnel to work with doctor in the operating room. Not only do such systems tend to be costly, they also tend to be far too complex to warrant broad adoption among orthopedic surgeons.

To overcome the accuracy and reliability issues associated with manual methods for determining joint placement parameters, while providing a cost-effective and relatively user-friendly approach that is unavailable in computer/robotically-assisted systems, a cost-effective, portable, and user-friendly tool and associated methods for measuring prosthetic component positioning would be advantageous. The presently disclosed prosthetic component positioning tool and associated methods for intra-operatively measuring prosthetic component placement parameters during orthopedic arthroplastic procedures are directed to overcoming one or more of the problems set forth above and/or other problems in the art.

### SUMMARY

According to one aspect, the present disclosure is directed to a method for estimating an orientation for placement of a prosthetic component. The method may comprise registration of anatomic reference plane(s). For example, in hip surgery this may comprise receiving, from an orientation sensor, information indicative of the orientation of a first virtual axis established between two pelvic landmarks such as the left and right anterior superior iliac spines. The method may also comprise calculating an orientation of a first virtual plane, the first virtual plane being perpendicular to the first virtual axis. The method may further comprise receiving, from the orientation sensor, information indicative of the orientation of a second virtual axis established between at least one of first two landmarks and third landmark such as the left or right pubic symphsis. The method may also comprise calculating an orientation of a second virtual plane based, at least in part, on the first virtual axis and the second virtual axis. The method may further comprise estimating an angle between an orientation sensor rigidly fixed on a placement tool for a prosthetic component and at least one of the first virtual plane or the second virtual plane.

In accordance with another aspect, the present disclosure is directed to a tool for placement of a prosthetic component relative to the anatomy of a patient. For example a tool for use in hip replacement surgery for placement of the acetabular cup component and registration of one or more pelvic planes. In one embodiment, the same tool performs the dual function of anatomic registration and component placement. This reduces the amount of hardware necessary and simplifies the user work flow. According to other embodiment, however, the anatomic registration and component placement functions may be implemented by separate tools performing dedicated functions. The tool may comprise an elongated linear member, a first end of which is configured for temporary attachment to a prosthetic component, and a second end of which is configured for application of force such as with a mallet. The tool may also comprise a first offset pointer to interface with first portion of a patient's anatomy and a second offset pointer to interface with a second portion of the patient's anatomy. The tool may also comprise an orientation sensor rigidly coupled to the elongated linear member and configured to detect information indicative of an orientation of the elongated linear member and the prosthetic component attached to it. The tool may further comprise a processor, communicatively coupled to the orientation sensor and configured to transmit the information indicative of the orientation of the elongated linear member to a remote device.

In accordance with another aspect, the present disclosure is directed to a system for estimating an orientation for placement of a prosthetic component relative to the anatomy of a patient. The system comprises an elongated tool, and an orientation sensor coupled to the tool and configured to detect information indicative of an orientation of the tool and any prosthetic component attached to it. The system also comprises a processor, communicatively coupled to the orientation sensor and configured to receive information indicative of the orientation of the elongated tool in a first position, the first position configured to estimate the orientation of a first virtual axis established between two anatomical landmarks such as the left and right anterior superior iliac spines of a patient's pelvis. The processor may also be configured to calculate an orientation of a first virtual plane, the first virtual plane being perpendicular to the first orientation. The processor may be further configured to receive information indicative of the orientation of the elongated tool in a second position. The second position is configured to estimate the orientation of a second virtual axis established between at least one of the estimated positions of the left and right anterior superior iliac spines and a third landmark such as the left or right pubic symphsis. The processor may also be configured to calculate an orientation of a second virtual plane based, at least in part, on the first virtual axis and the second virtual axis. The processor may be further configured to estimate an angle between the elongated tool and at least one of the first virtual plane or the second virtual plane.

### BRIEF DESCRIPTION OF THE DRAWINGS

FIG. 1 provides a front view of a portion of an exemplary hip joint, the type of which may be involved in a joint replacement procedure consistent with certain disclosed embodiments;

FIG. 2A provides a schematic view of exemplary components associated with a prosthetic hip joint, which may be used in a joint replacement procedure consistent with the disclosed embodiments;

FIG. 2B illustrates a magnified view of an exemplary prosthetic hip joint in a reduced state in accordance with certain disclosed embodiments;

FIG. 3 provides a diagrammatic view of an exemplary prosthetic cup positioning system (embodied as acetabular cup positioning system for intra-operative use during a total hip arthroplasty (THA) procedure) consistent with certain disclosed embodiments;

FIG. 4 provides a schematic view of exemplary components associated with a prosthetic cup positioning systems, such as the acetabular cup positioning system illustrated in FIG. 3;

FIG. 5A illustrates an exemplary position of tool 310 during a registration process that involves estimating an orientation of a first virtual plane associated with a virtual coordinate position associated with a pelvis, consistent with certain disclosure embodiments;

FIG. 5B illustrates an exemplary position of tool 310 during the registration process that involves estimating orientation of a second virtual plane associated with the virtual coordinate position associated with the pelvis, in accordance with certain disclosed embodiments;

FIG. 6 illustrates exemplary anatomical planes associated with the virtual coordinate system of the pelvis, the orientations of one or more of which may be estimated by processes consistent with the disclosed embodiments;

FIGS. 7A and 7B illustrate exemplary system for simultaneously registering a measurement tool sensor and a pelvic sensor, in accordance with certain disclosed embodiments;

FIG. 8 illustrates an exemplary embodiment of a user interface that may be provided on a monitor or output device for intra-operatively displaying the monitored prosthetic orientation parameters in real time, consistent with certain disclosed embodiments;

FIG. 9 provides a flowchart depicting an exemplary process to be performed by one or more processing devices associated with an exemplary prosthetic cup positioning system, consistent with certain disclosed embodiments; and

FIG. 10 provides a flowchart illustrating another exemplary process to be performed by one or more processing devices associated with an exemplary prosthetic component positioning system, in accordance with certain disclosed embodiments.

### DETAILED DESCRIPTION

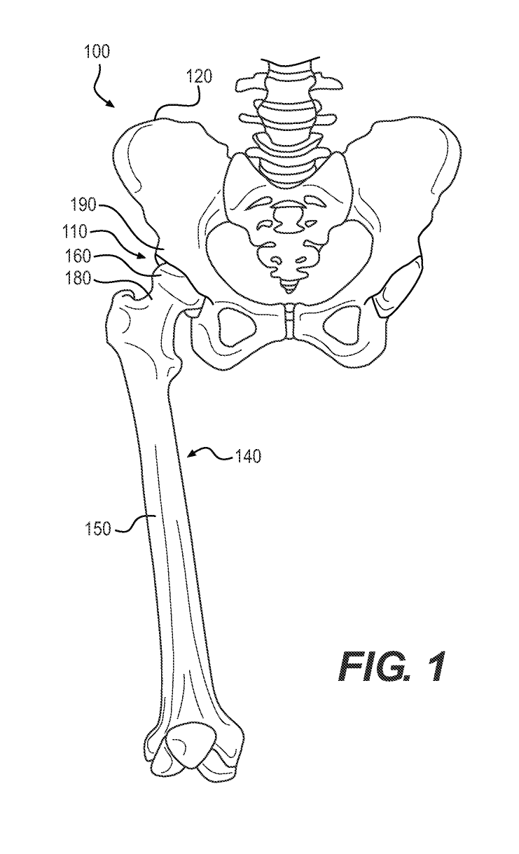

FIG. 1 illustrates a front view of an exemplary portion of the pelvic region 100 of the human body, which includes a hip joint 110. Proper articulation of hip joint 110 contributes to many basic structural and motor functions of the human body, such as standing and walking As illustrated in FIG. 1, hip joint 110 comprises the interface between pelvis 120 and the proximal end of femur 140. The proximal end of femur 140 includes a femoral head 160 disposed on a femoral neck 180. Femoral neck 180 connects femoral head 160 to a femoral shaft 150. Femoral head 160 fits into a concave socket in pelvis 120 called the acetabulum 190. Acetabulum 190 and femoral head 160 are both covered by articular cartilage (not shown) that absorbs shock and promotes articulation of hip joint 110.

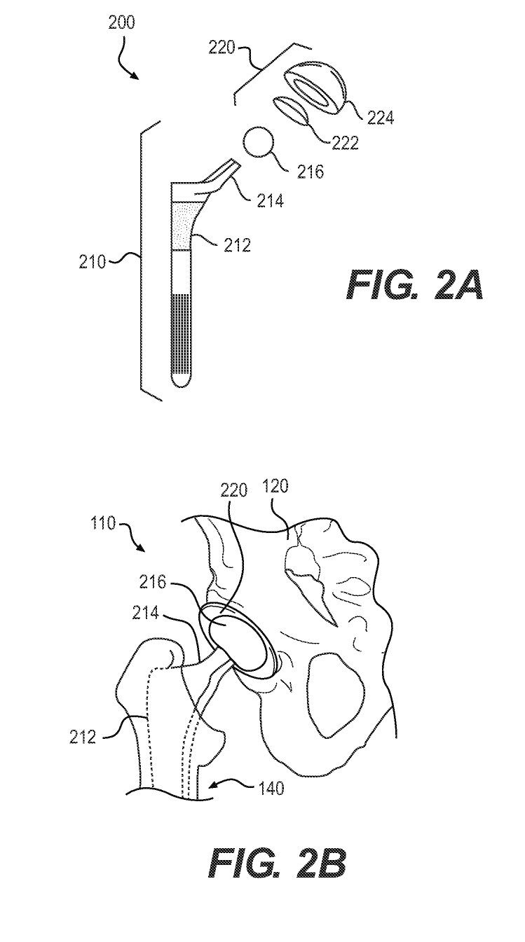

Over time, hip joint 110 may degenerate (due, for example, to osteoarthritis) resulting in pain and diminished functionality of the joint. As a result, a hip replacement procedure, such as total hip arthroplasty or hip resurfacing, may be necessary. During a hip replacement procedure, a surgeon may replace portions of hip joint 110 with artificial prosthetic components. For example, in one type of hip replacement procedure—called total hip arthroplasty (THA)—the surgeon may remove femoral head 160 and neck 180 from femur 140 and replace them with a femoral prosthesis. Similarly, the surgeon may resect or resurface portions of acetabulum 190 using a surgical reamer or reciprocating saw, and may replace the removed portions of acetabulum 190 with a prosthetic acetabular cup. Prosthetic components associated with the hip joint 110 are illustrated in FIG. 2A.

As illustrated in FIG. 2A, the natural (or “native”) femoral components removed during the arthroplasty may be replaced with a prosthetic femoral component 200 comprising a prosthetic head 216, a prosthetic neck 214, and a stem 212. Stem 212 of prosthetic femoral component 26 is typically anchored in a cavity that the surgeon creates in the intramedullary canal of femur 140.

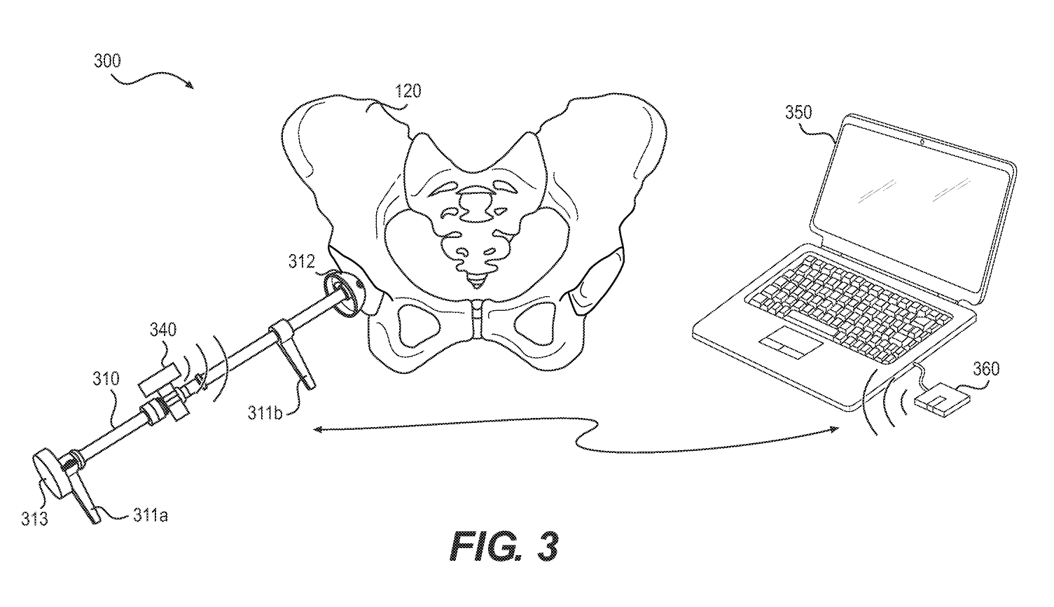

Similarly, the native acetabular components removed during the hip replacement procedure may be replaced with a prosthetic acetabular component 220 comprising a cup 224 that may include a liner 222. To install acetabular component 220, the surgeon connects cup 224 to a distal end (312 of FIG. 3) of an impactor tool (310 of FIG. 3) and implants cup 224 into the reamed acetabulum 190 by repeatedly applying force to a proximal end (313 of FIG. 3) of the impactor tool 310. If acetabular component 220 includes a liner 222, the surgeon snaps liner 222 into cup 224 after implanting cup 224 within acetabulum 220.

FIG. 2B illustrates a magnified view of an exemplary prosthetic hip joint in a reduced (i.e., assembled) state. As illustrated in FIG. 2B, the stem 212 is secured within the intramedullary canal of femur 140. The prosthetic head 216 is engaged with the acetabular component 220 of pelvis 120 to form the new prosthetic joint.

FIG. 3 provides a diagrammatic illustration of an exemplary system 300 for intra-operatively measuring prosthetic cup placement parameters during orthopedic arthroplastic procedures, such as a replacement procedure for hip joint 110. Those skilled in the art will recognize that embodiments consistent with the presently disclosed systems and methods may be employed in any environment involving arthroplastic procedures, such as the hip and shoulder.

For example, in accordance with the exemplary embodiment illustrated in FIG. 3, system 300 may embody a system for intra-operatively—and in real-time or near real-time—evaluating the placement orientations for a prosthetic cup, relative to a patient's anatomy. In particular, system 300 may provide a tool equipped with an orientation sensor and associated tracking system for determining the angles of abduction/adduction and/or anteversion/retroversion of the acetabular cup prior to permanent attachment of the cup to the patient's pelvis. To do so, however, the tool's orientation sensor must be registered to the patient's pelvic anatomy, the process for which will be described in further detail below. Individual components of exemplary embodiments of orthopedic placement monitoring system 300 will now be described in more detail.

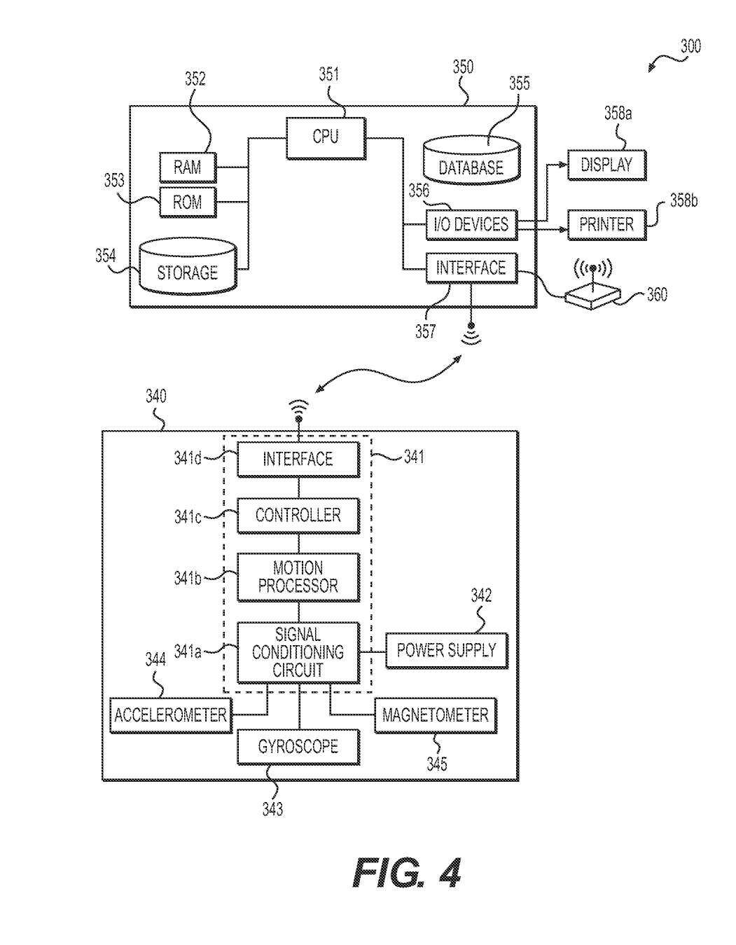

As illustrated in FIG. 3, system 300 may include a tool 310 equipped with at least one orientation sensor 340 for estimating an orientation of an acetabular cup 312 relative to the anatomy of the patient and a processing device (such as processing system 350 (or other computer device for processing data received by system 300)), and one or more wireless communication transceivers 360 for communicating with the orientation sensor 340 on tool 310 and one or more orientation sensors attached to the patient's anatomy (not shown). The components of system 300 described above are exemplary only, and are not intended to be limiting. Indeed, it is contemplated that additional and/or different components may be included as part of system 300 without departing from the scope of the present disclosure. For example, although wireless communication transceiver 360 is illustrated as being a standalone device, it may be integrated within one or more other components, such as processing system 350. Thus, the configuration and arrangement of components of system 300 illustrated in FIG. 4 are intended to be exemplary only.

Processing system 350 may include or embody any suitable microprocessor-based device configured to process and/or analyze information indicative of placement of a prosthetic component. According to one embodiment, processing system 350 may be a general purpose computer programmed with software for receiving, processing, and displaying information indicative of the orientation of tool 310 (which may be representative of the orientation of the prosthetic component attached to it). According to other embodiments, processing system 350 may be a special-purpose computer, specifically designed to communicate with, and process information for, other components associated with system 300. Individual components of, and processes/methods performed by, processing system 350 will be discussed in more detail below.

Processing system 350 may be communicatively coupled to an orientation sensor 340 (and any additional orientation sensors (not shown) used in system 300) and may be configured to receive, process, and/or analyze data measured by the orientation sensor 340. According to one embodiment, processing system 350 may be wirelessly coupled to orientation sensor 340 via wireless communication transceiver(s) 360 operating any suitable protocol for supporting wireless (e.g., wireless USB, ZigBee, Bluetooth, Wi-Fi, etc.) In accordance with another embodiment, processing system 350 may be wirelessly coupled to orientation sensor 340, which, in turn, may be configured to collect data from the other constituent sensors and deliver it to processing system 350. In accordance with yet another embodiment, certain components of processing system 350 (e.g. I/O devices 356) may be suitably miniaturized for fixation to tool 310 or integration with sensor 340.

Wireless communication transceiver(s) 360 may include any device suitable for supporting wireless communication between one or more components of system 300. As explained above, wireless communication transceiver(s) 360 may be configured for operation according to any number of suitable protocols for supporting wireless, such as, for example, wireless USB, ZigBee, Bluetooth, Wi-Fi, or any other suitable wireless communication protocol or standard. According to one embodiment, wireless communication transceiver 360 may embody a standalone communication module, separate from processing system 350. As such, wireless communication transceiver 360 may be electrically coupled to processing system 350 via USB or other data communication link and configured to deliver data received therein to processing system 350 for further processing/analysis. According to other embodiments, wireless communication transceiver 360 may embody an integrated wireless transceiver chipset, such as the Bluetooth, Wi-Fi, NFC, or 802.11x wireless chipset included as part of processing system 350.

Tool 310 may comprise an elongated member having a longitudinal axis that extends between a first end 312 and a second end 313. First end 312 may be configured for placing an acetabular cup, within an acetabulum 190 of pelvis 120. According to one exemplary embodiment, first end 312 may be configured for temporary rigid attachment to the acetabular cup prosthetic, such that the orientation of the tool 310 can be used to accurately represent the orientation of the acetabular cup relative to the patient's pelvis. As an example of temporary and rigid attachment, the first end 312 consists of an acetabular cup that is screwed on, allowing placement of the cup and removal of the tool after fixation of the cup in the pelvis. Tool 310 may also have a plurality of pointers 311a, 311b and an orientation sensor 340 coupled to a body of tool 310.

According to one embodiment, tool 310 may embody an acetabular cup placement device that is adapted to provide orientation measurements associated with tool 310. Acetabular cup placement device may include a first end 312 that is configured to temporarily and rigidly attach to a acetabular prosthetic cup that is to be impacted into a patient's acetabulum once the proper position of the acetabular cup has been determined. Acetabular cup placement device may include an impact surface at a second end 313 that provides a substantially flat surface upon which a surgeon can apply an impact force using a hammer or other object to drive the acetabular cup into position within the patient's pelvis 120. Although tool is illustrated in FIG. 3 as an impactor-type tool, it is contemplated that tool 310 may embody any type of elongated device that is configured to interact with a patient's acetabulum in order to emulate the position and orientation of the acetabular cup relative to a patient's pelvis.

Pointers 311a, 311b may include any structure(s) suitable for interfacing with a portion of a patient's anatomy to provide a uniform offset of tool 310 to the portion of the patient's anatomy. According to one embodiment, pointers 311a, 311b are sized and designed such that when they are placed on a flat surface the longitudinal axis of tool 310 is maintained parallel to the flat surface. As such, pointers 311a, 311b offset the longitudinal axis of the tool equally from the portions of the patient's anatomy that they are in contact with. According to one embodiment, at least one of the pointers is designed with a sliding mechanism so that the lateral distance between pointers 311a, 311b can be varied by sliding the pointer along the shaft of tool 310.

Orientation sensor 340 may be any system suitable for measuring information that can be used to accurately measure orientation in 3 dimensions. When affixed to tool 310, orientation sensor 340 may be configured to measure the orientation of the virtual longitudinal axis formed between a first end 312 and a second end 313 (or vice versa) of tool 310. Using the tool 310, orientation sensor 340 can be registered or calibrated to a virtual coordinate system associated with the patient's pelvic anatomy. Once registered, orientation sensor 340 can be used to measure the orientation of tool 310 and any prosthetic component attached to it relative to one or more planes associated with the patient's pelvic anatomy. According to one embodiment, at least one orientation sensor 340 is attached to or embedded within a portion of an elongated member 310 of a placement tool used during a hip replacement surgery. In another embodiment, at least one additional orientation sensor (not shown) is attached or embedded within a portion of the patient's pelvis and used in combination with orientation sensor 340 attached to tool 310, in order to account for changes in orientation of the patient's pelvis after registration and prior to cup placement.

FIG. 4 provides a schematic diagram illustrating certain exemplary subsystems associated with system 300 and its constituent components. Specifically, FIG. 4 is a schematic block diagram depicting exemplary subcomponents of processing system 350 and orientation sensor 340 in accordance with certain disclosed embodiments.

As explained, processing system 350 may be any processor-based computing system that is configured to receive placement parameters associated with an orthopedic joint 110, store anatomic registration information, analyze the received placement parameters to extract data indicative of the placement of prosthetic components of orthopedic joint 110 with respect to the patient's anatomy, and output the extracted data in real-time or near real-time. Non-limiting examples of processing system 350 include a desktop or notebook computer, a tablet device, a smartphone, wearable or handheld computers, or any other suitable processor-based computing system.

For example, as illustrated in FIG. 4, processing system 350 may include one or more hardware and/or software components configured to execute software programs, such as software tracking placement parameters associated with a prosthetic component of orthopedic joint 110 and displaying information indicative of the placement of the component. According to one embodiment, processing system 350 may include one or more hardware components such as, for example, a central processing unit (CPU) or microprocessor 351, a random access memory (RAM) module 352, a read-only memory (ROM) module 353, a memory or data storage module 354, a database 355, one or more input/output (I/O) devices 356, and an interface 357. Alternatively and/or additionally, processing system 350 may include one or more software media components such as, for example, a computer-readable medium including computer-executable instructions for performing methods consistent with certain disclosed embodiments. It is contemplated that one or more of the hardware components listed above may be implemented using software. For example, storage 354 may include a software partition associated with one or more other hardware components of processing system 350. Processing system 350 may include additional, fewer, and/or different components than those listed above. It is understood that the components listed above are exemplary only and not intended to be limiting.

CPU 351 may include one or more processors, each configured to execute instructions and process data to perform one or more functions associated with processing system 350. As illustrated in FIG. 4, CPU 351 may be communicatively coupled to RAM 352, ROM 353, storage 354, database 355, I/O devices 356, and interface 357. CPU 351 may be configured to execute sequences of computer program instructions to perform various processes, which will be described in detail below. The computer program instructions may be loaded into RAM 352 for execution by CPU 351.

RAM 352 and ROM 353 may each include one or more devices for storing information associated with an operation of processing system 350 and/or CPU 351. For example, ROM 353 may include a memory device configured to access and store information associated with processing system 350, including information for identifying, initializing, and monitoring the operation of one or more components and subsystems of processing system 350. RAM 352 may include a memory device for storing data associated with one or more operations of CPU 351. For example, ROM 353 may load instructions into RAM 352 for execution by CPU 351.

Storage 354 may include any type of mass storage device configured to store information that CPU 351 may need to perform processes consistent with the disclosed embodiments. For example, storage 354 may include one or more magnetic and/or optical disk devices, such as hard drives, CD-ROMs, DVD-ROMs, or any other type of mass media device. Alternatively or additionally, storage 314 may include flash memory mass media storage or other semiconductor-based storage medium.

Database 355 may include one or more software and/or hardware components that cooperate to store, organize, sort, filter, and/or arrange data used by processing system 350 and/or CPU 351. For example, database 355 may include historical data such as, for example, stored placement data associated with the orthopedic joint. CPU 351 may access the information stored in database 355 to provide a comparison between previous joint component placement and current (i.e., real-time) placement data. CPU 351 may also analyze current and previous placement parameters to identify trends in historical data. These trends may then be recorded and analyzed to allow the surgeon or other medical professional to compare the placement parameters with different prosthesis designs and patient demographics. It is contemplated that database 355 may store additional and/or different information than that listed above.

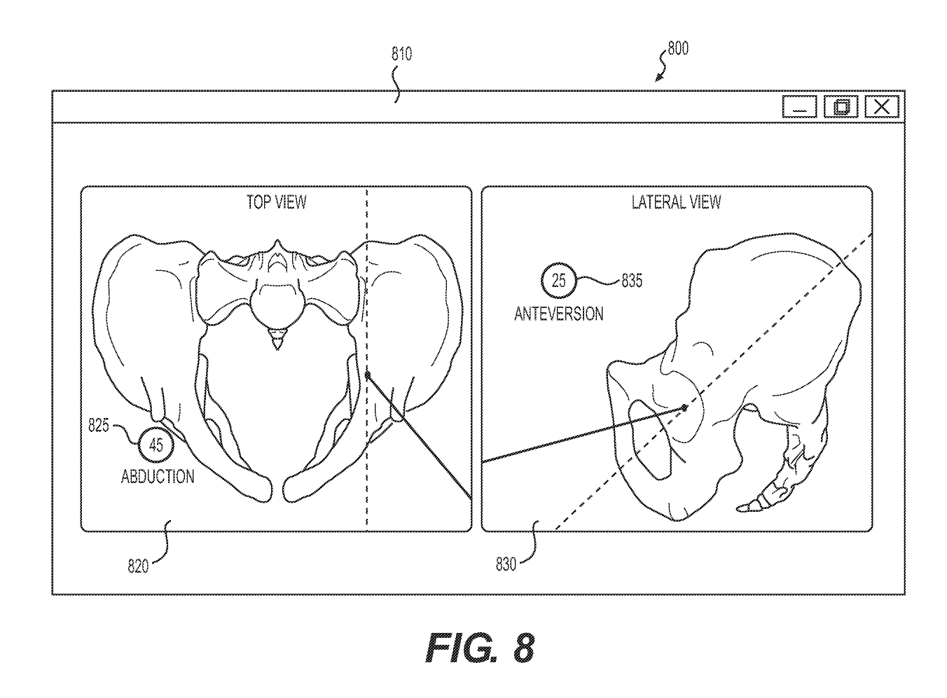

I/O devices 356 may include one or more components configured to communicate information with a user associated with system 300. For example, I/O devices may include a console with an integrated keyboard and mouse to allow a user to input parameters associated with processing system 350. I/O devices 356 may also include a display including a graphical user interface (GUI) (such as GUI 800 shown in FIG. 8) for outputting information on a display monitor 358a. In certain embodiments, the I/O devices may be suitably miniaturized and integrated with tool 310. I/O devices 356 may also include peripheral devices such as, for example, a printer 358b for printing information associated with processing system 350, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 357 may include one or more components configured to transmit and receive data via a communication network, such as the Internet, a local area network, a workstation peer-to-peer network, a direct link network, a wireless network, or any other suitable communication platform. For example, interface 357 may include one or more modulators, demodulators, multiplexers, demultiplexers, network communication devices, wireless devices, antennas, modems, and any other type of device configured to enable data communication via a communication network. According to one embodiment, interface 357 may be coupled to or include wireless communication devices, such as a module or modules configured to transmit information wirelessly using Wi-Fi or Bluetooth wireless protocols. Alternatively or additionally, interface 357 may be configured for coupling to one or more peripheral communication devices, such as wireless communication transceiver 320.

As explained, orientation sensor 340 may include one or more subcomponents configured to detect and transmit information that either represents 3-dimensional orientation or can be used to derive an orientation of the orientation sensor 340 (and, by extension, any object that is affixed relative to orientation sensor 340, such as an insertion or impactor tool 310 and any attached prosthetic component. Orientation sensor 340 may embody a device capable of determining a 3-dimensional orientation associated with any body to which orientation sensor 340 is attached. According to one embodiment, orientation sensor(s) 340 may be an inertial measurement unit including a microprocessor 341, a power supply 342, and one or more of a gyroscope 343, an accelerometer 344, or a magnetometer 345.

According to one embodiment, inertial measurement unit(s) 340 may contain a 3-axis gyroscope 343, a 3-axis accelerometer 344, and a 3-axes magnetometer 345. It is contemplated, however, that fewer of these devices with fewer axes can be used without departing from the scope of the present disclosure. For example, according to one embodiment, inertial measurement units may include only a gyroscope and an accelerometer, the gyroscope for calculating the orientation based on the rate of rotation of the device, and the accelerometer for measuring earth's gravity and linear motion, the accelerometer providing corrections to the rate of rotation information (based on errors introduced into the gyroscope because of device movements that are not rotational or errors due to biases and drifts). In other words, the accelerometer may be used to correct the orientation information collecting by the gyroscope. Similar the magnetometer 345 can be utilized to measure the earth's magnetic field and can be utilized to further correct gyroscope errors. Thus, while all three of gyroscope 343, accelerometer 344, and magnetometer 345 may be used, orientation measurements may be obtained using as few as one of these devices. The use of additional devices increases the resolution and accuracy of the orientation information and, therefore, may be advantageous when orientation accuracy is important.

As illustrated in FIG. 4, microprocessor 341 of inertial measurement unit 340 may include different processing modules or cores, which may cooperate to perform various processing functions. For example, microprocessor 341 may include, among other things, an interface 341d, a controller 341c, a motion processor 341b, and signal conditioning circuitry 341d. Controller 341c may be configured to control and receive conditioned and processed data from one or more of gyroscope 343, accelerometer 344, and magnetometer 345 and transmit the received data to one or more remote receivers. The data may be pre-conditioned via signal conditioning circuitry 341a, which includes amplifiers and analog-to-digital converters or any such circuits. The signals may be further processed by a motion processor 341b. Motion processor 341b may be programmed with so-called “motion fusion” algorithms to collect and process data from different sensors to generate error corrected orientation information. The orientation information may be a mathematically represented as an orientation or rotation quaternion, euler angles, direction cosine matrix, rotation matrix of any such mathematical construct for representing orientation known in the art. Accordingly, controller 341c may be communicatively coupled (e.g., wirelessly via interface 341d as shown in FIG. 4, or using a wireline protocol) to, for example, processing system 350 and may be configured to transmit the orientation data received from one or more of gyroscope 343, accelerometer 344, and magnetometer 345 to processing system 350, for further analysis.

Interface 341d may include one or more components configured to transmit and receive data via a communication network, such as the Internet, a local area network, a workstation peer-to-peer network, a direct link network, a wireless network, or any other suitable communication platform. For example, interface 341d may include one or more modulators, demodulators, multiplexers, demultiplexers, network communication devices, wireless devices, antennas, modems, and any other type of device configured to enable data communication via a communication network. According to one embodiment, interface 341d may be coupled to or include wireless communication devices, such as a module or modules configured to transmit information wirelessly using Wi-Fi or Bluetooth wireless protocols. As illustrated in FIG. 4, inertial measurement unit(s) 340 may be powered by power supply 342, such as a battery, fuel cell, MEMs micro-generator, or any other suitable compact power supply.

Importantly, although microprocessor 341 of inertial measurement unit 340 is illustrated as containing a number of discreet modules, it is contemplated that such a configuration should not be construed as limiting. Indeed, microprocessor 341 may include additional, fewer, and/or different modules than those described above with respect to FIG. 4, without departing from the scope of the present disclosure. Furthermore, in other instances of the present disclosure that describe a microprocessor are contemplated as being capable of performing many of the same functions as microprocessor 341 of inertial measurement unit 340 (e.g., signal conditioning, wireless communications, etc.) even though such processes are not explicitly described with respect to microprocessor 341. Those skilled in the art will recognize that many microprocessors include additional functionality (e.g., digital signal processing functions, data encryption functions, etc.) that are not explicitly described here. Such lack of explicit disclosure should not be construed as limiting. To the contrary, it will be readily apparent to those skilled in the art that such functionality is inherent to processing functions of many modern microprocessors, including the ones described herein.

Microprocessor 341 may be configured to receive data from one or more of gyroscope 343, accelerometer 344, and magnetometer 345 and transmit the received data to one or more remote receivers. Accordingly, microprocessor 341 may be communicatively coupled (e.g., wirelessly (as shown in FIG. 4, or using a wireline protocol) to, for example, processing system 350 and configured to transmit the orientation data received from one or more of gyroscope 343, accelerometer 344, and magnetometer 345 to processing system 350, for further analysis. As illustrated in FIG. 4, microprocessor 341 may be powered by power supply 342, such as a battery, fuel cell, MEMs micro-generator, or any other suitable compact power supply.

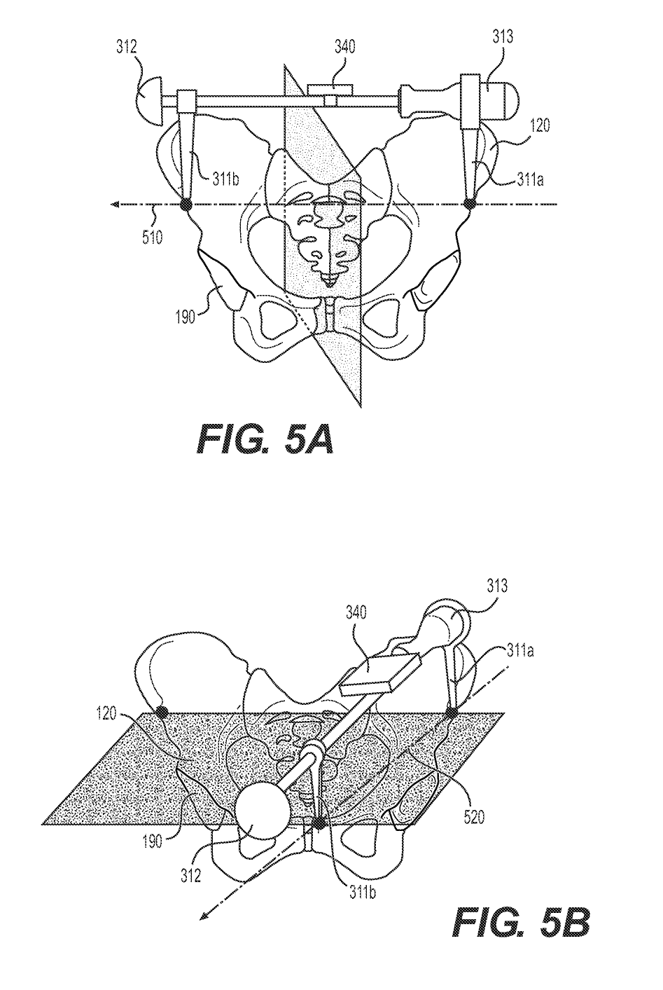

As explained, in order for system 300 to properly estimate the orientation of the prosthetic cup relative to pelvis 120, orientation sensor 340 must be registered to a virtual coordinate system of the patient's pelvis 120. Orientation sensor 340 has it's own X, Y, Z coordinate system and the process of registration establishes the relationship between the sensor's coordinate system and the patient's anatomy. The term “virtual,” as is used herein refers to a plane, vector, or coordinate system that exists as a mathematical or algorithmic representation within a computer software program. In other words, “virtual coordinate system” refers to an algorithmic mapping of points within an environment to a particular object, such as a bone or other portion of the patient's anatomy. To estimate the orientation and position of the prosthetic cup relative to pelvis 120, system 300 is configured to measure an orientation of the longitudinal axis of tool 310 using orientation sensor 340 in different positions relative to certain anatomical landmarks associated with the patient's pelvic anatomy. Using geometrical relationships associated with the anatomical landmarks, the information indicative of the orientation of tool 310 can be used to derive a virtual coordinate space that is representative of the pelvis, and associate orientation sensor 340 with this virtual coordinate space. FIGS. 5A, 5B, 6, and 9 illustrate an exemplary process for establishing a virtual coordinate space for the pelvis, registering orientation sensor 340 to the virtual coordinate space, and estimating a orientation for placement of a prosthetic cup in accordance with the disclosed embodiments.

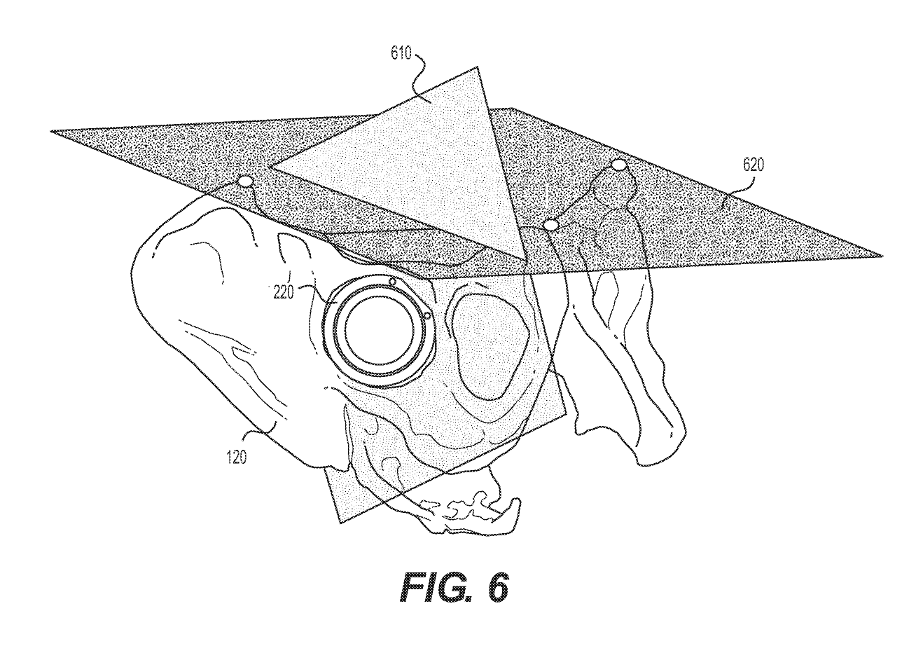

A common reference plane utilized for measurement of acetabular cup orientation is the anterior pelvic plane (illustrated as plane 620 of FIG. 6 and the plane of FIG. 5B) which is defined by the locations of the left and right anterior superior iliac spines (ASIS) and pubic symphsis. The saggital plane (illustrated as plane 610 in FIG. 6 and the plane of FIG. 5A) is perpendicular to the anterior pelvic plane. According to one embodiment, once orientation sensor 340 has been registered/calibrated to these anatomical planes of pelvis 120, orientation sensor 340 can be used to estimate the prosthetic cup orientation relative to the pelvis and, as such, can be used to determine the abduction and anteversion angles of the prosthetic cup relative to the saggital and anterior pelvic plane, respectively. Importantly, although the processes described in accordance with certain exemplary embodiments used the saggital and anterior pelvic planes to create a virtual coordinate system for the patient's anatomy, it is contemplated that any number of anatomical calibration techniques and landmarks can be used to determine the virtual pelvic coordinate space. For example, it is contemplated that certain bony landmarks of the pelvis can be used to determine the orientation of the transverse pelvic plane and this plan can be used as a basis for registering orientation sensor 340. Consequently, any of a number of different combinations of reference points/planes that can be used to define a virtual coordinate system of pelvis 120 and subsequently register sensor 340 to the virtual coordinate system without departing from the scope of the present disclosure.

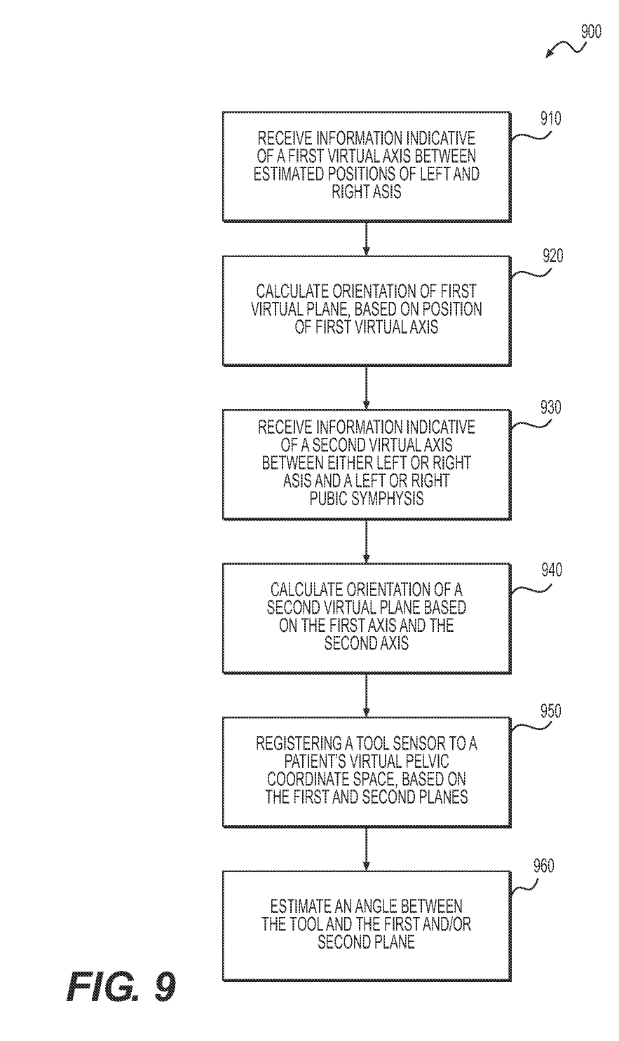

One exemplary process for registering orientation sensor 340 to a virtual coordinate system associated with pelvis 120 is illustrated in flowchart 900 of FIG. 9. As illustrated in FIG. 9, the registration process commences by receiving, at processing system 350 from orientation sensor 340, information indicative of a first orientation between estimated positions of left and right anterior superior iliac spines (ASIS) (Step 910, FIG. 9). FIG. 5A illustrates an exemplary embodiment for using tool 310 to measure the information indicative of the first orientation.

As illustrated in FIG. 5, pointers 311a, 311b of tool 310 are placed at portions of the patient's anatomy that correspond to the left and right ASIS of pelvis 120. In this position, the inertial measurement unit 340 measures the orientation associated with tool 310 which corresponds to the orientation of a virtual axis that passes through the 2 ASIS's. During a surgical procedure, pointers 311a, 311b are brought in contact with a patient's anatomy corresponding to estimated positions of the anatomical landmarks of pelvis 120 (in an exemplary embodiment, the left and right anterior superior iliac spines (ASIS)). When the user is satisfied with the position of pointers 311a, 311b, the orientation associated with tool 310 is measured by inertial measurement unit 340 and transmitted to processing system 350 for storage. One or more points or vectors maybe be recorded and averaged to improve accuracy. The recorded orientation is parallel to the frontal horizontal axis of the body (axis that passes from side to side). Using mathematical formulas based on geometry the processing unit is then able to calculate the orientation of a plane that is perpendicular to this recorded orientation (Step 920, FIG. 9). This perpendicular plane is parallel to saggital plane (610 of FIG. 6) and its orientation is indicative of the orientation of saggital plane 610.

As illustrated in FIG. 9, the registration process continues by receiving, at processing system 350 from an orientation sensor 340, information indicative of the orientation of a second virtual axis established between at least one of the estimated positions of the left and right anterior superior iliac spines and one of left or right pubic symphsis (Step 930, FIG. 9). As illustrated in FIG. 5B, for example, pointers 311a, 311b are placed on one of the left or right ASIS and one of the left or right pubic symphsis. In this position, orientation sensor 340 measures the orientation of a second virtual axis that passes through those points. The orientation of this axis relative to the axis recorded in Step 910 is calculated. Since the three anatomic landmarks used in the registration process lie on the anterior pelvic plane, the two virtual axes recorded in steps 910 and 920 are parallel to the anterior pelvic plane (and non-parallel to one another). The orientation of the anterior pelvic plan can be therefore be calculated by the processing unit using mathematical formulas based on geometry (Step 940, FIG. 9).

According to the exemplary embodiment, once the first virtual plane (indicative of a plane parallel with saggital plane 610) and the second virtual plant (indicative of a plane parallel with the anterior pelvic plane 620) have been determined, processing system 350 registers/calibrates orientation sensor 340 to the patient's virtual pelvic coordinate space (Step 950, FIG. 9) and stores that information. According to one embodiment, processing system 350 is configured to mathematically transform the raw orientation measurements from orientation sensor 340 of tool 310 to an orientation angle relative to either or both of the first and second virtual planes (Step 960, FIG. 9). When first end 312 of tool 310 is engaged with acetabulum 190 of pelvis 120, the orientation information detected by orientation sensor 340 may be used to estimate the orientation of a prosthetic cup relative to one or more virtual planes associated with the pelvic anatomy of the patient. For example, the angle formed by the longitudinal axis of tool 310 with the first virtual plane associated with pelvis 120 represents the angle of the prosthetic cup relative to the saggital plane, and is indicative of the angle or amount of abduction and/or adduction of the prosthetic cup relative to the hip joint. According to another exemplary embodiment, the angle formed by the longitudinal axis of tool 310 with the second virtual plane associated with pelvis 120 represents the angle of the prosthetic cup relative to the anterior pelvic plane, and is indicative of the angle or amount of an anteversion and/or retroversion of the prosthetic cup relative to the hip joint.

As illustrated in the embodiments illustrated in FIGS. 3, 5A, and 5B, tool 310 may be embodied in several different forms. For example, as illustrated in FIG. 3, tool 310 may be configured as a registration and impactor placement tool that can be used by a surgeon to register anatomic planes and impact prosthetic cup 220 into pelvis 120 of the patient. Alternatively or additionally, and as illustrated in FIGS. 5A and 5B, tool 310 may be configured as an placement tool alone and used in conjunction with another elongated standalone registration tool. In this alternate embodiment of the system, the standalone registration tool is equipped with an orientation sensor similar to sensor 340 and pointers similar to 311a, 311b but does not attach to a prosthetic component and the placement tool is equipped with orientation sensor 340 but not pointers 311a, 311b and attaches to the prosthetic component. Those skilled in the art will appreciate that tool 310 may embody any longitudinally elongated device to which orientation sensor 340 and, additionally in the case of an exemplary embodiment, pointers 311a, 311b can be coupled can be used or retrofitted to function as tool 310.

Although certain exemplary embodiments do not rely on any pre-operative or intra-operative imaging data, certain embodiments consistent with the present disclosure may be used in conjunction with such information. For example, if the surgeon is unable to reliably find and point to the bony landmarks (e.g. in the case of an obese patient) imaging data (such as x-ray or CT scan data) can be used to aid in completing the above-outlined registration process. For example, although the ASIS landmarks are easily and reliably found even in obese patients, palpating and pointing to the pubic symphsis can be challenging. In such situations, instead using the pubic symphsis to determine the second plane, pelvic tilt data (i.e., the tilt associated with the anterior pelvic plane with respect to the frontal (coronal) plane of the body) may be determined using imaging techniques (such as a lateral X-ray) either pre-operatively or intra-operatively. Any other imaging modality such as MRI and CT-scan may also be used to get this information. This pelvic tilt information may be input to the processing unit and this along with the first registration of the ASIS is sufficient to determine the orientation of the anterior pelvic plane, without having to palpate and/or point to the pubic symphsis.

In accordance with one exemplary embodiment, an orientation sensor may be registered to the virtual coordinate system associated with the pelvis and used to detect the orientation of the pelvis. The real-time orientation measurements of the pelvis along with the tool orientation measurements by orientation sensor 340 is used by processor 350 to calculate the correct prosthetic cup orientation.

According to one exemplary embodiment, the pelvis sensor (which may be embodied as a second inertial measurement unit similar to inertial measurement unit in orientation sensor 340) may be placed anywhere on the acetabulum either in the surgically exposed area or through the skin. The pelvis sensor housing can be rigidly attached to the bone using pins or screws that are commonly used in orthopedic surgery, such as, for example, a Steinmann pin.

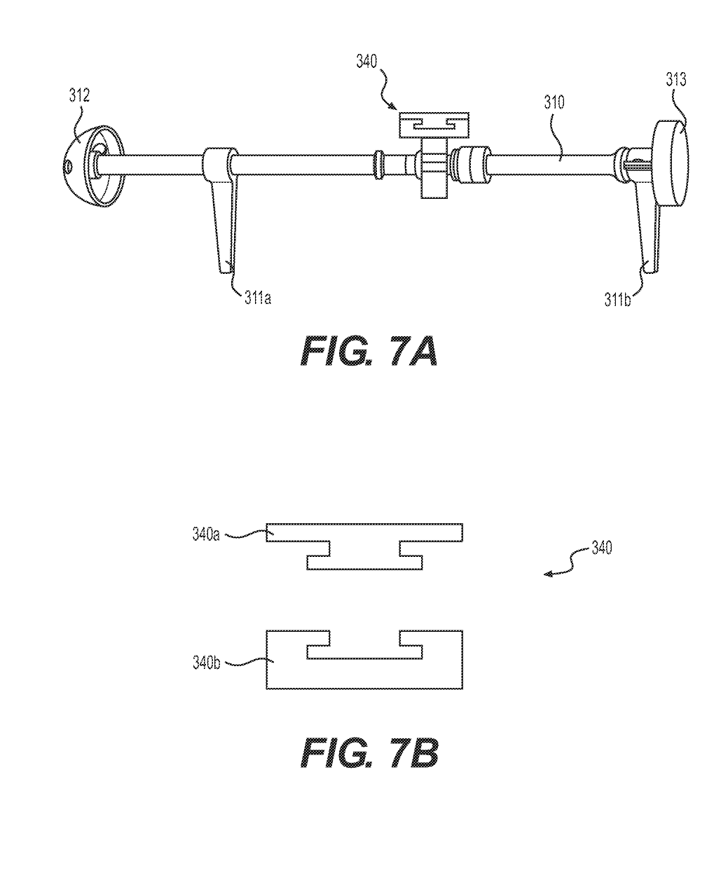

For accurate measurement of pelvis orientation, the pelvis sensor may be registered/calibrated to the virtual coordinate system of the pelvis. This can be done using a direct method where the pelvis orientation sensor is oriented along bony landmarks using a procedure similar to the one described for registering orientation sensor 310. In such direct registration methods, the pelvis sensor may be removably coupled to tool 310 and registered to the anatomic plane concurrently with the placement tool sensor 340. FIGS. 7A and 7B illustrate an embodiment in which a second orientation sensor 340a is coupled to tool 310 (via a slideable connection associated with a first orientation sensor 340b that is coupled to tool 310). Such removable connection simplifies the procedure by allowing the pelvis sensor (second orientation sensor 340a) to be registered to the pelvic coordinate system simultaneously with the registration of first orientation sensor 340b. As illustrated in FIGS. 7A and 7B, the pelvis sensor housing is designed such that it is removable (e.g. sliding groove mechanism on the bottom surface of the that mates with the top surface of the insertion tool sensor housing allowing the first and second orientation sensors 340b, 340a to be stacked on top of each other). After the registration process, the second orientation sensor 340a is disengaged from the housing and rigidly affixed to the acetabulum. After the fixation process, processing system 350 is prompted to record the orientation of the pelvis sensor as the reference starting position for the pelvis 120. The processing system 350 is then able to measure the pelvis orientation relative to the anatomic planes independently and concurrently with the measurement of the orientation of the insertion tool and calculate the correct orientation of the tool and attached prosthetic component with respect to the pelvis.

According to another embodiment, the pelvis sensor may be registered/calibrated using a standalone elongate registration tool, in a similar manner to which orientation sensor 340 on tool 310 is registered.

An indirect method of registration can also be utilized to register/calibrate the pelvis sensor. In this embodiment, the relationship between the X, Y, Z coordinate frames of tool sensor 340b and pelvis sensor 340a is established. Once this virtual relationship is established, then it is only necessary to register the tool sensor 340b, since the registration process establishes the relationship between insertion tool sensor 340b and the virtual pelvic coordinate system and the relationship between 340a and the same virtual pelvic coordinate system can be derived based on the pre-established relationship between 340b and 340a.

One skilled in the art will recognize that here are many ways to establish the relationship between the coordinate frames of the insertion tool and pelvis sensors. One method is to measure their orientations when there is a known orientation relationship between them. For example, the sensors could come out of the box removably fixed on a base plate with a known orientation between them. When the sensors report their orientation in this arrangement the relationship between their coordinate frames can be established. Another similar method is to stack the sensors using a method similar to that described earlier. The sensors could come pre-stacked on the insertion tool out of the box. In this case the work flow would include a first measurement to establish the relationship between the sensors. After this the pelvis sensor would be removed and placed on pelvis. The insertion tool with the insertion tool sensor only affixed to it would then be used to register the virtual pelvic coordinate system.

FIG. 8 provides an exemplary screen shot 800, respectively, corresponding to a graphical user interface (GUI) 810 associated with processing system 350. As illustrated in screen shot 800, GUI 810 may include a first user interface element 820 that is configured to display, in real-time or near-real time, the angle of orientation of the prosthetic cup relative to the saggital plane. According to one embodiment, user interface element 820 may provide a numerical gauge 825 that displays the angle of the prosthetic cup with relative to the saggital plane. This angle is referred to as abduction and/or adduction angle. Alternatively or additionally, user interface element 820 may provide a graphical representation of a pelvic bone, a graphical representation of a virtual plane parallel to the saggital plane, and a graphical representation of an axis of the prosthetic component indicating the real-time position of the prosthetic cup, as calculated by processor 350.

Alternatively or additionally, GUI 810 may include user interface element 830 may provide a second numerical gauge 835 that displays the angle of the prosthetic cup with the anterior pelvic plane. This angle is referred to as anteversion and/or retroversion angle. Alternatively or additionally, user interface element 820 may provide a graphical representation of a pelvic bone, a graphical representation of a virtual plane parallel to the anterior pelvic plane, and a graphical representation of an axis of the prosthetic component indicating the real-time position of the prosthetic cup, as measured by tool 310.

As an alternative or in addition to a prosthetic cup placement system that can be used on a general population of patients, certain embodiments consistent with the present disclosure contemplate patient specific instruments for assisting with the placement of the prosthetic cup. The invention can also be used in conjunction with patient specific instruments/guides. Patient-specific instruments typically utilize pre-operative 3D imaging of the patient's anatomy. 3D models of the hip joint based on the images are created and a pre-operative surgical plan is created. Based on the surgical plan, patient specific instrumentation to assist the surgeon in achieving the surgical objectives is created. These instruments typically have matching/interlocking features that are representative of the inverse of the patient's anatomic features and/or other such patient specific features. These features allow fixation of the patient-specific instrumentation onto the patient's bone during surgery such that a pre-determined orientation of the instruments relative to the patient's anatomy is established.

In one embodiment of the current invention, a reference sensor is embedded into or attached to a patient specific instrument. The orientation of the reference sensor with respect to the patient specific instrument is known either from design or measured during the manufacturing process of the patient-specific instrument. Alternatively, the reference sensor can be attached to the patient-specific instrument intra-operatively at a known orientation using mating features on the patient-specific instrument or alignment marks. Also, as previously mentioned, the patient specific instrument is designed for fixation to the patient's anatomy at a pre-determined anatomic orientation. With the above two relationships known, the relative orientation of the reference sensor with respect to the patient's anatomy can then be derived. In effect the reference sensor is pre-operatively registered to the patient's anatomy using the patient-specific instrument as a vehicle (regardless of when the reference sensor is actually attached). Such “pre-registration” eliminates the need for manual registration of the anatomic plane and results in a system this is truly “point and shoot.”

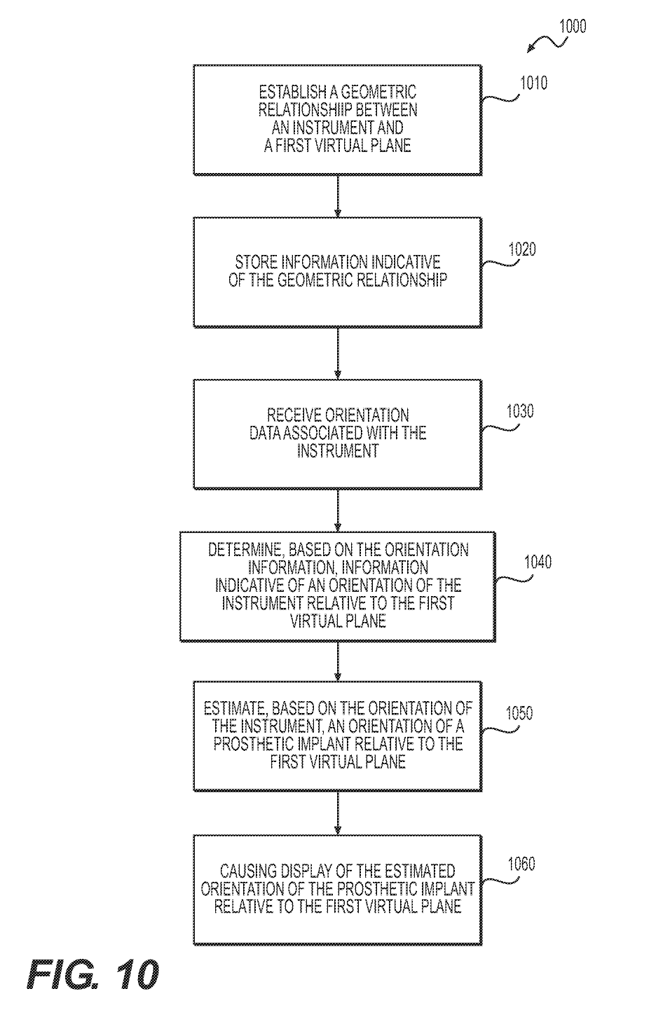

Processes and methods consistent with the disclosed embodiments have been described in accordance with specific joint replacement procedures, namely a hip joint replacement procedure. This skilled in the art will recognize, however, that these descriptions were exemplary only, and that the presently disclosed prosthetic placement tracking system—using a technique that involves either manual tool registration/calibration with a patient's anatomy or a patient-specific registration technique—can be used in most any situation in which precise placement of a prosthetic component is important. Indeed, although certain embodiments were described with respect to tracking placement of a prosthetic acetabular cup in a patient's pelvis, it is contemplated that such methods and systems are equally applicable to other joints, such as shoulder joint. FIG. 10 provides a flowchart 1000 illustrating a more generalized process for intra-operatively tracking prospective component placement using a surgical system, similar to 300.

As illustrated in flowchart 1000 of FIG. 10, the process involves establishing a geometric relationship between an instrument equipped with an orientation sensor and a first virtual plane (Step 1010). The geometric relationship may include or embody the algorithms necessary to convert raw orientation data associated with the surgical instrument into position or orientation information relative to the virtual pelvic coordinate system. As explained, the geometric relationship may be established by manually calibrating the sensor on the surgical instrument, such as orientation sensor 340, to a virtual plane associated with a patient's anatomy. For example, as outlined above with respect to FIG. 9, the geometric relationship between sensor 340 and a saggital plane of a patient's body can be established by measuring and storing the orientation between the left and right anterior superior iliac spines (ASIS) of a patient's pelvis. Processing system 350 can then mathematically derive the orientation of the saggital plane (or a parallel thereto) as a plane orthogonal to the measured orientation.

In a similar way, during a shoulder reconstruction or replacement procedure, sensor 340 can be manually registered to a virtual plane or other feature associated with the patient's shoulder anatomy. For example, one or more bony landmarks associated with the patient's torso, spine, shoulders, or upper body may be used to register orientation sensor 340 to one or more of the saggital, transverse, or coronal planes (or one or more planes parallel thereto). The geometric relationship(s) between the surgical instrument and virtual plane(s) and instrument may be stored by processing system 350 (Step 1020), for future tracking of instrument 310 relative to the virtual plane.

According to certain embodiments, the sensor on the instrument may be mapped to multiple planes associated with the patient's anatomy, depending upon the desired tracking capability of the instrument. For example, the sensor on the instrument may be mapped to a first virtual plane parallel to the saggital plane of the patient's body in order to track the abduction/adduction of the joint (or associated prosthetic components). Alternatively or additionally, the sensor on the instrument may be mapped to a second virtual plane parallel to the coronal plane of the patient's body in order to track the anteversion/retroversion of the joint (or associated prosthetic components).

Once the geometric relationships between the instrument sensor (i.e., sensor 340) and the virtual anatomical planes have been established, real-time orientation information associated with the instrument can be received/collected (Step 1030). As explained, tool 310 may be fitted with one or more orientation sensors 340 comprising of inertial measurement units configured to collect raw inertial data from at least one gyroscope 343, accelerometer 344, and/or magnetometer 345 that may be embedded within inertial measurement unit.

Based on this raw inertial measurement data, processing system 350 may determine information indicative of an orientation of the instrument relative to one or more of the established virtual anatomic planes (Step 1040), and estimate the orientation of the prosthetic implant relative to these virtual planes (Step 1050). In particular, because processing system 350 can use the stored algorithms corresponding to the geometric relationships between the instrument and the virtual anatomical planes to transform the raw orientation data of the instrument into the virtual anatomic space. Using this transformation, the orientation of the instrument relative to the anatomical planes can be calculated and processed for display (Step 1060), in a GUI associated with processing system 350.

It will be apparent to those skilled in the art that various modifications and variations can be made to the disclosed systems and methods for measuring orthopedic parameters associated with a reconstructed joint in orthopedic arthroplastic procedures. Other embodiments of the present disclosure will be apparent to those skilled in the art from consideration of the specification and practice of the present disclosure. It is intended that the specification and examples be considered as exemplary only, with a true scope of the present disclosure being indicated by the following claims and their equivalents.

What is claimed is:

1. A method for estimating an orientation for placement of a prosthetic implant, comprising:
receiving, from an orientation sensor, information indicative of an orientation of a first virtual axis established between estimated positions of left and right anterior superior iliac spines of a patient's pelvis;
calculating an orientation of a first virtual plane, the first virtual plane being perpendicular to the first virtual axis;
receiving, from the inertial measurement unit, information indicative of an orientation of a second virtual axis established between at least one of the estimated positions of the left and right anterior superior iliac spines and one of left or right pubic symphsis;
calculating an orientation of a second virtual plane based, at least in part, on the first virtual axis and the second virtual axis; and
estimating an angle between the orientation sensor and at least one of the first virtual plane or the second virtual plane.

2. The method of claim 1, wherein calculating the orientation of the second virtual plane includes calculating the orientation of the second virtual plane as the plane that contains the first virtual axis and the second virtual axis.

3. The method of claim 1, wherein the first virtual plane includes at least one of the saggital plane or a plane parallel to the saggital plane.

4. The method of claim 1, wherein the second virtual plane includes at least one of the anterior pelvic plane or a plane parallel to the anterior pelvic plane.

5. The method of claim 1, wherein estimating the angle includes estimating the angle between the orientation sensor and the first virtual plane and the second virtual plane, wherein the angle between the orientation sensor and the first virtual plane is indicative of at least one of abduction or adduction of the prosthetic implant relative to the pelvis, and wherein the angle between the orientation sensor and the second virtual plane is indicative of at least one of the anteversion or retroversion of the prosthetic implant relative to the pelvis.

6. The method of claim 1, further comprising receiving, from a second orientation sensor, information indicative of an orientation of the pelvis, wherein the orientations of the first and second virtual plane are based, at least in part, on the orientation of the pelvis.

7. The method of claim 1, further comprising causing display of the estimated angle between the orientation sensor and at least one of the first virtual plane or the second virtual plane.

8. A tool for estimating an orientation for placement of a prosthetic cup relative to a pelvis of a patient, the tool comprising:
an elongated linear member, a first end of which is configured to engage a first portion of a patient's anatomy;
an orientation sensor coupled to the elongated linear member and configured to detect information indicative of an orientation of the elongated linear member; and
a processor, communicatively coupled to the orientation sensor and configured to transmit the information indicative of the orientation of the elongated linear member to a remote device.

9. The tool of claim 8, further comprising:
a first pointer coupled the elongated linear member and configured to provide an offset between the first portion and the first end; and
a second pointer coupled to the elongated linear member and configured to provide an offset between a second portion of the patient's anatomy and a second end of the elongated linear member.

10. The tool of claim 9, wherein the lengths of the first pointer and the second pointer provide a substantially uniform offset at the first end and the second end.

11. The tool of claim 9, wherein the elongated linear member is an impactor tool for installing an acetabular cup in the pelvis of the patient.

12. The tool of claim 9, wherein at least one of the first or second pointers is slidably coupled to the elongated linear member, such that the distance between the first pointer and the second pointer is adjustable.

13. The tool of claim 8, wherein the orientation sensor is an inertial measurement unit that includes at least one of a gyroscope, an accelerometer, or a magnetometer.

14. The tool of claim 8, wherein the inertial measurement unit includes a gyroscope and an accelerometer.

15. The tool of claim 8, further including a second orientation sensor, removably coupled to at least a portion of the elongated member and adapted for coupling to at least a portion of the pelvis and, when registered to a pelvic anatomy of the patient, configured to detect information indicative of the orientation of the pelvis.

16. A system for estimating a position for placement of a prosthetic implant relative to a bone of a patient, the system comprising:
an elongated probe tool;
an orientation sensor coupled to the elongated probe tool and configured to detect information indicative of an orientation of the elongated probe tool;
a processor, communicatively coupled to the orientation sensor and configured to:
receive information indicative of the orientation of the elongated probe tool in a first position, the first position configured to estimate the orientation of a first virtual axis established between left and right anterior superior iliac spines of a patient's pelvis;
calculate an orientation of a first virtual plane, the first virtual plane being perpendicular to the first virtual axis;
receive information indicative of the orientation of the elongated probe tool in a second position, the second position configured to estimate the orientation of a second virtual axis between at least one of the estimated positions of the left and right anterior superior iliac spines and one of left or right pubic symphsis;
calculate an orientation of a second virtual plane based, at least in part, on the first virtual axis and the second virtual axis; and
estimate an angle between the orientation sensor and at least one of the first virtual plane or the second virtual plane.

17. The system of claim 16, further comprising:
a first pointer coupled to the elongated linear member and configured to provide an offset between the first portion and the first end; and
a second pointer coupled to the elongated linear member and configured to provide an offset between the second portion and the second end.

18. The system of claim 17, wherein the lengths of the first pointer and the second pointer provide a substantially uniform offset at the first end and the second end.

19. The system of claim 17, wherein the elongated linear member is an impactor tool for installing an acetabular cup in the pelvis of the patient.

20. The system of claim 17, wherein at least one of the first or second pointers is slidably coupled to the elongated linear member, such that the distance between the first pointer and the second pointer is adjustable.

21. The system of claim 16, wherein calculating the orientation of the second virtual plane includes calculating the orientation of the second virtual plane as the plane that contains the first virtual axis and the second virtual axis.

22. The system of claim 16, wherein the first virtual plane includes at least one of the saggital plane or a plane parallel to the saggital plane.

23. The system of claim 16, wherein the second virtual plane includes at least one of the anterior pelvic plane or a plane parallel to the anterior pelvic plane.

24. The system of claim 16, further comprising a display device, wherein the processor is further configured to cause display of the estimated angle between the orientation sensor and at least one of the first virtual plane or the second virtual plane.

25. The system of claim 16, wherein the orientation sensor includes at least one inertial measurement unit that includes at least one of a gyroscope, an accelerometer, or a magnetometer.

26. The system of claim 16, wherein the orientation sensor includes at least one inertial measurement unit that includes a gyroscope and an accelerometer.

27. A method for estimating an orientation for placement of a prosthetic implant, comprising:
establishing a geometric relationship between an instrument and a first virtual plane, the first virtual plane associated with at least a portion of an anatomy of a patient;
storing information indicative of the geometric relationship;
receiving orientation data associated with the instrument;
determining, based on the orientation information and the established geometric relationship, information indicative of the orientation of the instrument relative to the first virtual plane;
estimating, based, at least in part, on the orientation of the instrument, an orientation of the prosthetic implant relative to the first virtual plane; and
causing display of the estimated position of the prosthetic implant relative to the first virtual plane.

28. The method of claim 27, wherein establishing the geometric relationship includes:
receiving, from an orientation sensor associated with the instrument, information indicative of the orientation of a first virtual axis established between estimated positions of a first pair of points on the at least a portion of the anatomy; and
calculating an orientation of a first virtual plane based, at least in part, on information indicative of the first virtual axis.

29. The method of claim 28, wherein the first pair of points includes a first point associated with an estimated positions of left anterior superior iliac spine of the patient's pelvis and a second point associated with an estimated positions of right anterior superior iliac spine of the patient's pelvis, wherein the orientation of the first virtual plane is calculated as a plane perpendicular to the first virtual axis.

30. The method of claim 28, wherein the first pair of points includes between at least one of the estimated positions of the left and right anterior superior iliac spines and one of left or right pubic symphsis, wherein the orientation of the first virtual plane is calculated as a plane containing the first virtual axis and one of the left of or right pubic symphsis.

31. The method of claim 27, wherein the first virtual plane is the saggital plane or a plane parallel to the saggital plane of the patient's body.

32. The method of claim 27, wherein the first virtual plane is the anterior pelvic plane or a plane parallel to the anterior pelvic plane of the patient's body.

33. The method of claim 27, wherein the first virtual plane is the transverse plane or a plane parallel to the transverse plane of the patient's body.

34. The method of claim 27, further including:
establishing a second geometric relationship between the instrument and a second virtual plane;
receiving second orientation data associated with the instrument;
determining, based on the second orientation information and the established second geometric relationship, information indicative of an orientation of the instrument relative to the second virtual plane; and
estimating, based, at least in part, on the orientation of the instrument, an orientation of the prosthetic implant relative to the second virtual plane.

35. The method of claim 34, wherein establishing the second geometric relationship includes:
receiving, from the orientation sensor associated with the instrument, information indicative of a second virtual axis established between estimated positions of a second pair of points associated with a second portion of the anatomy; and
calculating an orientation of a second virtual plane based, at least in part, on information indicative of the first virtual axis and the second virtual axis.
